(19) United States
(12) Patent Application Publication
(10) Pub. No.: US 2010/0100059 A1
(43) Pub. Date: Apr. 22, 2010

(54) Methods and Apparatus for Preparing a Hemodialytic Site

(76) Inventors: Vialine Coleman, Atco, NJ (US); Samson Coleman, Atco, NJ (US)

Correspondence Address: Law Offices of Rita C. Chipperson, P.C., 55 Madison Ave, Suite 400, Morristown, NJ, 07960, US

(21) Appl. No.: 12/465,060

(22) Filed: May 13, 2009

Related U.S. Application Data
(60) Provisional application No. 61/196,208, filed on Oct. 16, 2008.

(51) Int. Cl. A61M 19/00

(52) U.S. Cl. 604/290; 604/308

(57) ABSTRACT

Methods and apparatus for preparing a hemodialytic site and protecting skin disorders and wounds. The apparatus includes a substantially rectangular pad formed of a transparent, non-absorbent, waterproof material. The pad is fastened to the site via two fasteners, each of the fasteners including hook and loop portions, each of the hook portions affixed along a longitudinal edge of the pad, a proximal end of the loop portion adhered to a proximal end of a respective one of the hook portions, and the loop portions substantially parallel to each other and aligned with a respective one of the hook portion. When preparing a hemodialytic site, the following steps are performed: applying a topical anesthesia to the hemodialytic site; positioning the apparatus over the hemodialytic site, fastening the apparatus to an area of a body surrounding the hemodialytic site; wearing the apparatus for a predetermined time period; and removing the apparatus.

### CROSS-REFERENCE TO RELATED APPLICATIONS

This application claims the benefit of the U.S. provisional patent application entitled “Samson Site Patch”, having Ser. No. 61/196,208, filed Oct. 16, 2008, which is hereby incorporated by reference in its entirety as if fully set forth herein.

### BACKGROUND OF THE INVENTION

Embodiments of the present invention generally relate to methods and apparatus for preparing a hemodialytic site. More specifically, the present invention relates to methods and apparatus for preparing a hemodialytic site via application of a topical anesthesia to the site previous to a hemodialytic procedure.

Diabetes affects an estimated 75 million Americans and that figure is predicted to increase over the upcoming years. Diabetes is a life long disease, and many diabetes patients receive specialty care for kidney disease. This care often includes periodic hemodialytic treatment. Medical practitioners insert intravenous catheters through a patient's skin in order to perform hemodialysis. Pain is caused upon insertion of the intravenous catheter, which can reduce cooperativity of patients in addition to causing anxiety and other negative effects on the well-being of the patient. To combat this problem, prior to hemodialytic treatment the site is often anesthetized via a topical anesthetic with an occlusive dressing.

Various devices for creating an occlusive dressing are known in the art. A traditional adhesive dressing is often used to cover the site that includes a flexible backing coated with adhesive and an inabsorbant sterile pad to cover the site. Typically, these types of bandages are inefficient because the adhesive loses adhesion when it comes in contact with the anesthesia.

Other devices for applying a topical anesthetic can take a number of forms such as a glove, a sock, or a tube. Such devices typically fit the body part snugly and hold the anesthesia against the skin. This type of device is affixed to the body via a hook and loop closure. Variations of this method are known in which a holder for anesthetic is that provided that is prefilled with anesthetic and holds the topical anesthetic against the patient's skin when applied.

Another apparatus for wrapping a member of the body to anesthetize or protect a medical site includes a penile circumcision dressing. Such a dressing includes a sleeve bandage with a plurality of straps extending outward with attachment apparatus on their distal ends. The bandage is secured by diametrically wrapping the straps around the sleeve and engaging the fasteners on the distal ends with the outside surface of the bandage. This type of dressing often includes the addition of a topical anesthetic in order to anesthetize the skin at the site.

### BRIEF SUMMARY OF THE INVENTION

Briefly stated, in one aspect of the present invention, an apparatus for preparing a hemodialytic site is provided. This apparatus includes: a pad, the pad formed of a transparent, non-absorbent, waterproof material; the pad being substantially rectangular; and two fasteners, each of said fasteners including a hook portion and a loop portion, each of the hook portions being substantially rectangular, each of the hook portions affixed along a longitudinal edge of said pad and extending throughout an entire length of the pad; the hook portions substantially parallel to each other, each of the loop portions being substantially rectangular, a proximal end of the loop portion adhered to a proximal end of a respective one of said hook portions; the loop portions substantially parallel to each other and aligned with the respective ones of the hook portions.

In another aspect of the present invention, a method for preparing a hemodialytic site is provided. This method includes the following steps: applying a topical anesthesia to the hemodialytic site; positioning an apparatus over the hemodialytic site; fastening the apparatus to an area of a body surrounding the hemodialytic site; wearing the apparatus for a predetermined time period; and removing the apparatus. The apparatus includes: a pad, the pad formed of a transparent, non-absorbent, waterproof material; the pad being substantially rectangular; and two fasteners, each of the fasteners including a hook portion and a loop portion, each of the hook portions being substantially rectangular, each of the hook portions affixed along a longitudinal edge of the pad and extending throughout an entire length of the pad; the hook portions substantially parallel to each other, each of the loop portions being substantially rectangular, a proximal end of the loop portion adhered to a proximal end of a respective one of the hook portions; the loop portions substantially parallel to each other and aligned with the respective ones of the hook portions.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is a perspective view of an apparatus for preparing a dialytic site while in use in accordance with one embodiment of the present invention;

FIG. 2 is a top view of the apparatus of FIG. 1 in accordance with one embodiment of the present invention;

FIG. 3 is a bottom view of the apparatus of FIG. 1 in accordance with one embodiment of the present invention; and

FIG. 4 depicts a flowchart of the steps of a process for preparing a dialytic site in accordance with one embodiment of the present invention.

### DETAILED DESCRIPTION OF THE INVENTION

Certain terminology may be used in the following description for convenience only and is not limiting. The words “lower” and “upper” and “top” and “bottom” designate directions in the drawings to which reference is made. The terminology includes the words above specifically mentioned, derivatives thereof and words of similar import.

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a strap” includes a plurality of straps. Thus, for example, a reference to “a method” includes one or more methods, and/or steps of the type described herein and/or which will become apparent to those persons skilled in the art upon reading this disclosure.

Although any methods and materials similar or equivalent to those described herein can be used in the practice or testing of the present invention, the preferred methods, constructs and materials are now described. All publications mentioned herein are incorporated herein by reference in their entirety.

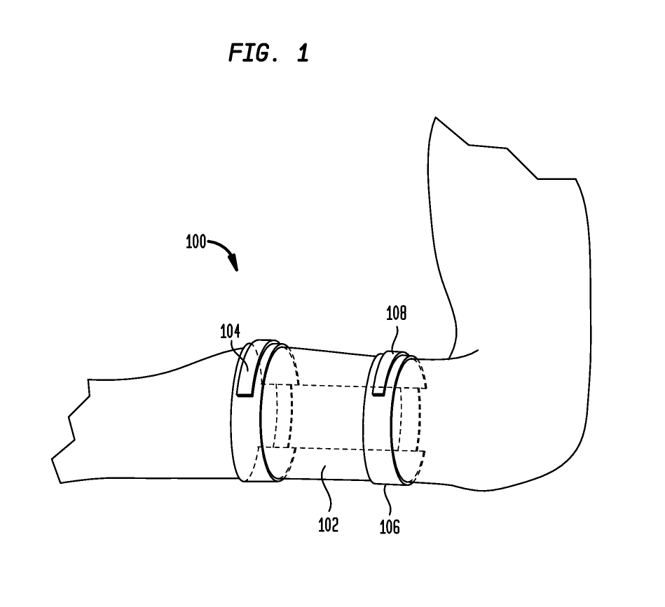

Referring first to FIG. 1, depicted is an in use view of an apparatus for preparing a dialytic site in accordance with one embodiment of the present invention. Apparatus 100 includes, inter alia, pad 102, a plurality of straps 104, and a plurality of fasteners 106. Apparatus 100 is designed to facilitate the application of a topical anesthesia to a hemodialytic site previous to a hemodialytic procedure in order to relieve pain caused by the procedure. Apparatus 100 is also designed to lie flat against the skin in a convenient and comfortable manner, thereby easing the lifestyle of patients forced to undergo periodic dialysis due to diseases such as Diabetes and the like.

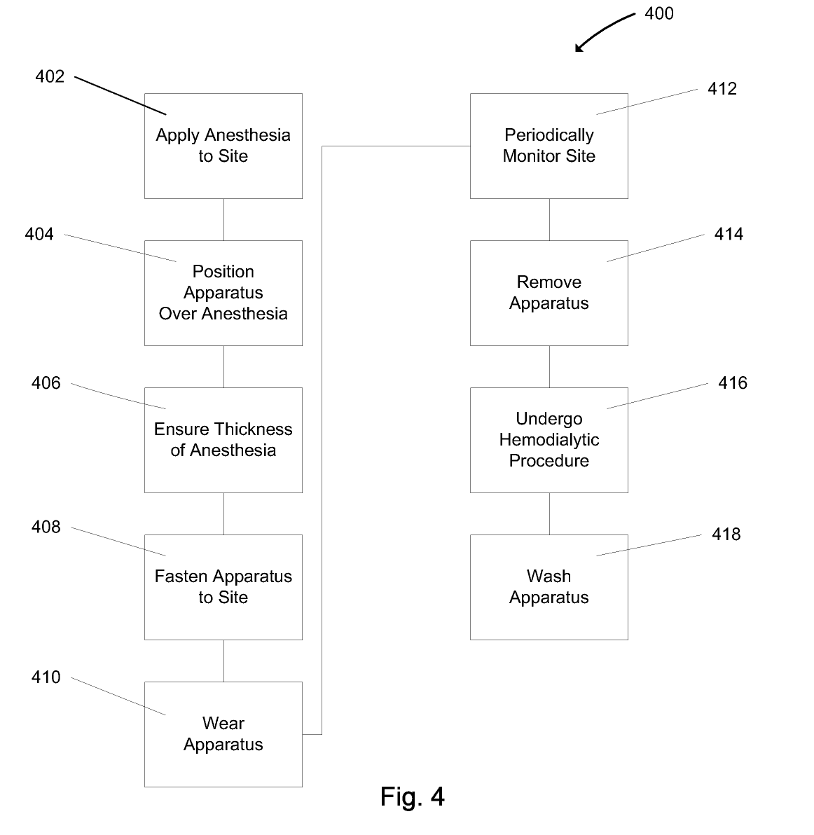

In addition to preparing a hemodialytic site, apparatus 100 may also be used to protect or heal skin disorders or wounds, surgical site incisions, and/or other skin maladies including, but not limited to, dry skin, scratched skin, broken skin, cut skin, and the like. When used for such purposes, apparatus 100 may be used as described with respect to FIG. 4 below except that the topical anesthesia is omitted (i.e., apparatus 100 is placed directly on the skin) or another ointment or the like may be substituted for the topical anesthesia (e.g., moisturizer, antibiotic cream, etc.). As also discussed in greater detail below with respect to FIG. 4, the use of apparatus 100 to isolate the skin and/or ointment from the surrounding environment prevents contaminants such as dirt, germs, bacteria, and fungus from contacting the skin in order to minimize the potential of an infection at the site of the skin disorder.

In the depicted embodiment of the present invention, apparatus 100 is applied to the forearm as shown and as described in greater detail below with respect to FIG. 4, however, alternate embodiments of the present invention are envisioned in which apparatus 100 is applied to areas of the body other than the forearm.

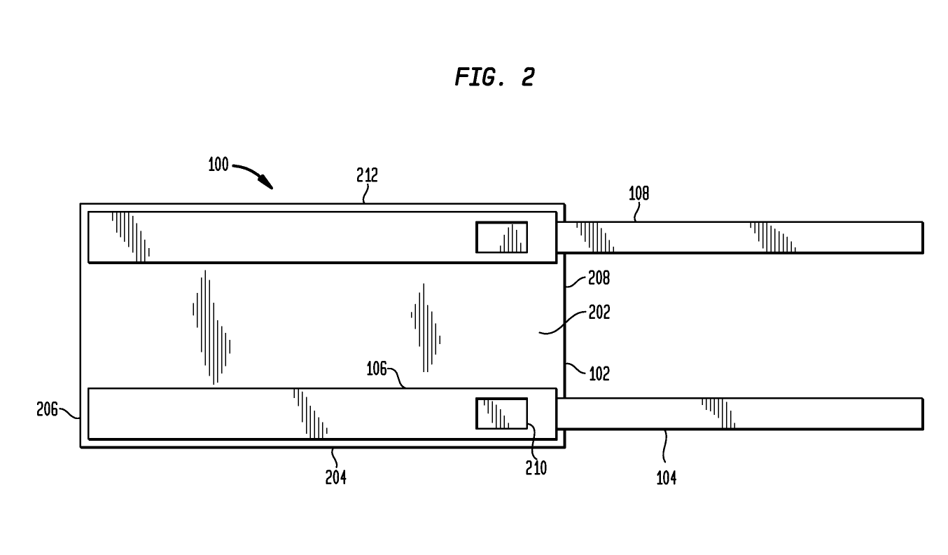

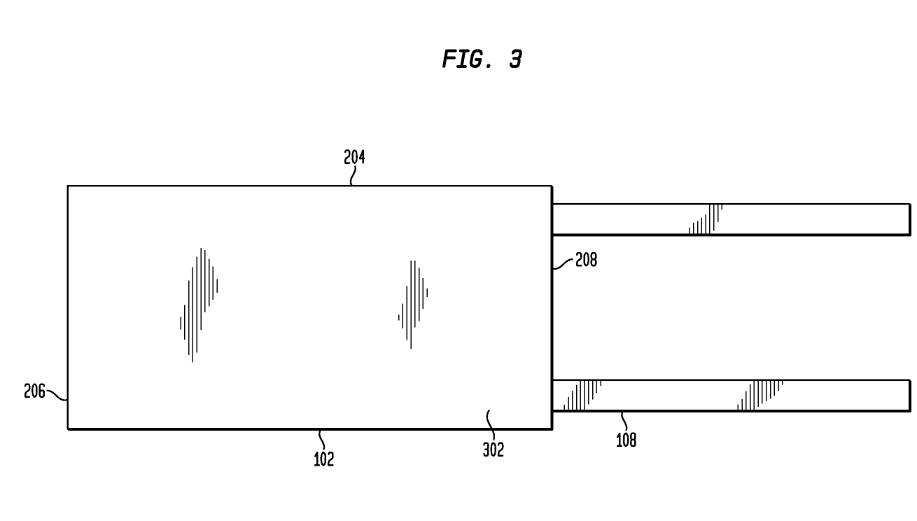

Referring now to FIGS. 2 and 3, depicted are top and bottom views, respectively, of apparatus 100. As shown, pad 102 is substantially rectangular in shape and has inwardly facing surface 202 and outwardly facing surface 302. In one embodiment of the present invention, pad 102 is a thin, single ply, plastic sheet manufactured from a transparent, non-absorbent, waterproof material, such as latex-free single ply thin plastic. This material is soft, safe, and flexible, thereby providing a more secure and comfortable fit for the user. The use of such a material, inter alia, minimizes the potential that pad 102 will adhere to the site of the skin disorder, thereby maximizing the comfort of the user. Although a latex-free material such as latex-free single ply thin plastic is used in the preferred embodiment to accommodate patients who may be allergic to latex, other materials can be substituted without departing from the scope of the present invention, including, but not limited to single ply thin plastic containing latex.

Although the embodiment of the present invention depicted in FIGS. 1-3 has a single ply pad 102, pads having multiple layers may be substituted without departing from the scope of the present invention. For example, in one embodiment of the present invention, pad 102 includes at least two plies. The two plies are adhered to each other along edges 206, 208, and 210. Edges 204 are not adhered to each other. This configuration provides a pad 102 in the form of a pocket. This pocket may be used for storage of small items during use of apparatus 100.

In some embodiments of the present invention, the length of pad 102 is varied to accommodate users with arms of varying diameters. For example, pad 102 of an apparatus 100 created for a child may have a shorter length than pad 102 of an apparatus 100 created for an adult since a child's arm has a smaller diameter and less length is required to encircle a significant portion of the outer circumference of a child's arm as compared to the length required to encircle a significant portion of an adult's arm. Although pad 102 is depicted in FIGS. 1-3 as substantially rectangular, it may be manufactured in varying shapes and sizes.

The transparent nature of pad 102 allows a user to view the hemodialytic site as it is being prepared for hemodialysis in accordance with methods such as that discussed in greater detail below with respect to FIG. 4. Allowing a user to view the hemodialytic site as the apparatus is secured to the user's body allows the user to more accurately position apparatus 100. For example, in some embodiments of the present invention, it is best to locate the hemodialytic site at the vertical and horizontal midpoints of pad 102. This positioning ensures maximum coverage of the hemodialytic site by pad 102. Furthermore, after apparatus 100 is properly positioned, the transparency of apparatus 100 allows the user to visually monitor the hemodialytic site during the anesthetization period, thereby facilitating detection of any skin irritation (e.g., redness, swelling, etc.) or other medical issues without removal of apparatus 100. However, alternate embodiments of the present invention are envisioned in which pad 102 is not transparent.

Additionally, in the embodiment of the present invention depicted in FIGS. 1-3, pad 102 is made of a non-absorbent, waterproof material. These features increase the efficiency of apparatus 100 by ensuring that the topical anesthesia applied to inwardly facing surface 202, as discussed in greater detail below with respect to FIG. 4, is not absorbed into pad 102. Rather, all of the topical anesthesia must be applied to the skin, thereby resulting in increased effectiveness of the topical anesthesia. This feature of the invention also facilitates proper use of apparatus 100 as it is easier for a user to determine and apply the proper quantity of topical anesthesia by eliminating the guesswork associated with determining how much anesthesia may have been absorbed into pad 102. Rather, the non-absorbent nature of pad 102 ensures that all of the topical anesthesia is applied to the hemodialytic site. These features also facilitate reuse of apparatus 100 as it may be washed and dried after use. However, alternate embodiments of the present invention are envisioned in which pad 102 is made of an absorbent material.

Attached to inwardly facing surface 202 are a plurality of fasteners 106. Each fastener 106 includes a hook portion 106 and a loop portion 108. Hook portion 106 is substantially rectangular and is at least partially adhered along the longitudinal edges 204 and 212 of inwardly facing surface 202 of pad 102. In the depicted embodiment in which pad 102 is substantially rectangular, two hook portions 106 are adhered or otherwise affixed along both longitudinal edges 204 and 212 such that they are substantially parallel to each other and to longitudinal edges 204 and 212. In this embodiment, hook portions 106 also extend along the entire longitudinal edge from distal latitudinal edge 206 to proximal latitudinal edge 208. This placement of hook portions 106 maximizes and centralizes the surface area of inwardly facing surface 202 that is unencumbered by hook portions 106. This feature facilitates application of the topical anesthesia as well as viewing of the hemodialytic site.

Additionally, extension of hook portions 106 along the entire length of pad 102 allows apparatus 100 to be adjusted to a greater quantity of diameters during use as loop portions 108 may be affixed to any point along hook portions 106. This feature allows a single size of apparatus 100 to accommodate a greater number of users having varying arm diameters. However, although hook portions 106 are depicted as substantially horizontal and extending along the full length of pad 102, varying shapes and sizes of hook portions 106 may be substituted without departing from the scope of the present invention.

Loop portions 108 are designed to mate with hook portions 106. One example of a suitable hook and loop type fastener 104 is Velcro®. This material tends to withstand wear and tear when apparatus 100 is re-used. Additionally, it provides a very secure fastening that minimizes the possibility of apparatus 100 falling off of the site. However, other types of Velcro® and/or non-Velcro® materials may be substituted for fastener 104 without departing from the scope of the present invention.

In the embodiment of the present invention depicted in FIGS. 1-3, each loop portion 108 is substantially rectangular and is at least partially adhered along longitudinal edges 204 and 212 of inwardly facing surface 202. In the depicted embodiment in which pad 102 is substantially rectangular, two loop portions 108 are adhered or otherwise affixed to both longitudinal edges 204 and 212 such that they extend significantly beyond proximal latitudinal edge 208, they are substantially parallel to each other, and they are in alignment with hook portions 106. Specifically, in the depicted embodiment, the proximal end of each loop portion 108 passes under the proximal end of its respective hook portion 106 and through slit 210, wherein slit 210 is located within hook portion 106 as depicted in FIG. 2. Thereafter, the proximal end of loop portion 108 is adhered or otherwise affixed to hook portion 106 by affixing the outwardly facing side of such end to the inwardly facing adjacent side of hook portion 106. In some embodiments, the outwardly facing side of loop portion 108 adjacent inwardly facing side 202 is also adhered or otherwise affixed to inwardly facing side 202, however, this is not required to achieve fasteners 104 of the present invention. This placement of loop portions 108 maximizes the length to which loop portions 108 extend beyond proximal latitudinal edge 208, thereby allowing apparatus 100 to be adjusted to a greater quantity of diameters during use as loop portions 108 may be affixed to any point along hook portions 106. This feature allows a single size of apparatus 100 to accommodate a greater number of users having varying arm diameters. However, although loop portions 108 are depicted as substantially horizontal and extending significantly beyond proximal latitudinal edge 208, varying shapes and sizes of loop portions 108 may be substituted without departing from the scope of the present invention.

The fasteners 104 of the present invention are designed to be easily operated via one hand. Since hemodialytic sites are commonly located on a user's forearm, it is desirable to include a fastener that may be easily operated with one hand as the hand associated with the forearm of the intended hemodialytic site is rendered unavailable during attachment of apparatus 100. Although fasteners 104 are depicted in FIGS. 1-3 as hook and loop type fasteners, other types of fasteners may be substituted without departing from the scope of the present invention.

In one embodiment of the present invention such as that depicted in FIGS. 1-3, apparatus 100 is offered in small, medium, and large sizes. In such an embodiment, the size of pad 102 is three (3) inches wide by six and one-half (6.5) inches long. In this same embodiment, the size of hook portions 106, respectively, are as follows: three-quarters (¾) of an inch wide by fifteen (15) inches long; three-quarters (¾) of an inch wide by twenty (20) inches long; and three-quarters (X) of an inch wide by twenty-eight (28) inches long. Additionally, in this same embodiment, the size of loop portions 108, respectively, are as follows: three-quarters (¾) of an inch wide by fifteen (15) inches long; three-quarters (¾) of an inch wide by twenty (20) inches long; and three-quarters (¾) of an inch wide by twenty-eight (28) inches long. However, alternate sizes may be substituted without departing from the scope of the present invention.

Referring lastly to FIG. 4, depicted is a flowchart of the steps of a method for preparing a hemodialytic site using an apparatus such as apparatus 100 in accordance with one embodiment of the present invention. Process 400 starts at 402, at which a topical anesthesia is applied to the user's skin at the location of the intended hemodialytic site. The anesthesia is typically applied after the skin has been washed well with soap and water and thoroughly dried. It is typically applied in accordance with the manufacturer's directions. For example, in some embodiments of the present invention, the topical anesthesia is a eutectic mixture of local anesthetics (“EMLA”). EMLA is typically sold in the form of an ointment and may be applied via squeezing the EMLA from a tube directly onto the skin. Alternatively, EMLA is sometimes sold with an applicator which is used to smooth the EMLA onto the skin at the hemodialytic site. However, non-EMLA topical anesthesia and other application methods may be substituted without departing from the scope of the present invention.

Process 400 then proceeds to 404 at which the user positions the apparatus such as apparatus 100 over the topical anesthesia. During this step, the transparency of the pad (e.g., pad 102) of the apparatus facilitates proper positioning over the topical anesthesia as it allows the user to view the anesthesia as the apparatus is positioned. It is important to ensure that the topical anesthesia will be entirely covered by the pad once the apparatus is secured to the arm or other body location. The majority of topical anesthesia requires the use of an occlusive dressing to isolate the topical anesthesia from the surrounding environment as this is a critical element in anesthetizing the hemodialytic site. In addition, the isolation of the topical anesthesia prevents contaminants such as dirt, germs, bacteria, and fungus from contacting the skin in order to minimize the potential of an infection at the hemodialytic site. Once the pad is properly positioned relative to the intended hemodialytic site, process 400 proceeds to 406.

At 406, the user ensures proper thickness of the topical anesthesia. This process is facilitated via the user's ability to view the intended hemodialytic site through pad 102. The proper thickness of the topical anesthesia, as is typically recommended by the manufacturer of the topical anesthesia, is important for proper anesthetization of the intended hemodialytic site. That is, the applied layer should be relatively thick in order to ensure maximum anesthetization.

Next, at 408, once the apparatus has been properly positioned and the proper thickness of topical anesthesia has been achieved, the user fastens the apparatus to secure it to the arm. When using the apparatus depicted in FIGS. 1-3, this fastening is achieved via attachment of loop fasteners 108 to hook fasteners 106 at a location in which the apparatus is snugly held to the arm or other portion of the body intended to be the hemodialytic site. The attachment of the apparatus must be tight enough to retain the apparatus in position yet loose enough to prevent squeezing of the topical anesthesia such that the thickness of same is decreased. Fasteners 104, as discussed above, are easily manipulated with one hand and may easily be readjusted until the proper fit is achieved.

Process 400 then proceeds to 410 at which the user begins wearing the apparatus. In a typical embodiment, a user will wear the apparatus for a duration of one to two hours or as otherwise recommended by the manufacture of the topical anesthesia. The unique design of the present invention allows the user to move and perform daily tasks without fear that the apparatus will reposition or be removed from the intended hemodialytic site. The pad and fasteners of the apparatus of the present invention provide a secure fit while also providing flexibility that allows a user to be mobile during the period in which anesthetization is occurring.

Process 400 then proceeds to 412 at which the user periodically monitors the intended hemodialytic site through the pad of the apparatus. Due to the transparent nature of the pad, the user has the ability to monitor the hemodialytic site without removing the apparatus. This is beneficial because during the anesthetization process, the possibility exists that the site may become irritated. If irritation such as inflammation, soreness, redness, etc. is observed through pad 102 during the anesthetization process, the user may prematurely remove the apparatus prior to completion of anesthetization to avoid further irritation until the problem with the topical anesthesia is diagnosed and corrected. Alternatively, if no adverse reaction is observed, the user continues to wear the apparatus for the specified time continually monitoring the intended hemodialytic site throughout.

Once the anesthetization time period has expired, process 400 proceeds to 414. At 414, the user removes the apparatus. For example, if apparatus 100 as depicted in FIGS. 1-3 is utilized, removal requires separation of the hook and loop portions of the fastening system.

Process 400 then proceeds to 416 at which the user undergoes the hemodialytic procedure at the hemodialytic site prepared using the methods and apparatus of the present invention. Finally, process 400 optionally proceeds to 418 at which the user may wash the apparatus for future use. This step is possible when the apparatus has a washable pad, such as the non-absorbent pad 102 of apparatus 100 as depicted in FIGS. 1-3. Reuse of the apparatus is optional, as it could also be discarded and a new apparatus utilized for each subsequent anesthetization.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An apparatus for preparing a hemodialytic site comprising:
a pad, said pad formed of a transparent, non-absorbent, waterproof material; said pad being substantially rectangular; and
two fasteners, each of said fasteners including a hook portion and a loop portion, each of said hook portions being substantially rectangular, each of said hook portions affixed along a longitudinal edge of said pad and extending throughout an entire length of said pad; said hook portions substantially parallel to each other, each of said loop portions being substantially rectangular, a proximal end of said loop portion adhered to a proximal end of a respective one of said hook portions; said loop portions substantially parallel to each other and aligned with said respective ones of said hook portions.

2. An apparatus according to claim 1, wherein said pad includes a pocket.

3. A method for preparing a hemodialytic site comprising the steps of:
applying a topical anesthesia to said hemodialytic site;
positioning an apparatus over said hemodialytic site, said apparatus including:
a pad, said pad formed of a transparent, non-absorbent, waterproof material; said pad being substantially rectangular; and
two fasteners, each of said fasteners including a hook portion and a loop portion, each of said hook portions being substantially rectangular, each of said hook portions affixed along a longitudinal edge of said pad and extending throughout an entire length of said pad; said hook portions substantially parallel to each other, each of said loop portions being substantially rectangular, a proximal end of said loop portion adhered to a proximal end of a respective one of said hook portions; said loop portions substantially parallel to each other and aligned with said respective ones of said hook portions;
fastening said apparatus to an area of a body surrounding said hemodialytic site;
wearing said apparatus for a predetermined time period; and
removing said apparatus.

4. An apparatus according to claim 3 further comprising the step of:
periodically monitoring said hemodialytic site during said wearing of said apparatus.

5. An apparatus according to claim 3 further comprising the steps of:
washing said apparatus after said removing of said apparatus; and
reusing said apparatus.

6. An apparatus according to claim 3 further comprising the step of:
adjusting a thickness of said topical anesthesia prior to said fastening.
